(19) United States
(12) Patent Application Publication
(10) Pub. No.: US 2012/0065412 A1
(43) Pub. Date: Mar. 15, 2012

(54) SYSTEM AND PROCESS FOR PRODUCING HIGHER-VALUE HYDROCARBONS FROM METHANE

(75) Inventors: Radwan Abdallah, Ludwigshafen (DE); Torsten Mäurer, Lambsheim (DE); Gerhard Theis, Maxdorf (DE)

(73) Assignee: BASF SE, Ludwigshafen (DE)

(21) Appl. No.: 13/321,129

(22) PCT Filed: May 10, 2010
(86) PCT No.: PCT/EP2010/056318

§ 371 Date: Nov. 17, 2011

(30) Foreign Application Priority Data
May 20, 2009 (EP) 09160749.9

(51) Int. Cl. C07D 301/03; C07C 31/08; B01J 19/00; C07C 47/02; C07C 67/00; C07C 17/00; C07C 2/00; C07C 47/06

(52) U.S. Cl. 549/518; 585/326; 585/319; 568/840; 568/449; 560/261; 570/216; 570/241; 422/600; 422/187

(57) ABSTRACT

The present invention relates to a process for producing hydrocarbons from methane, which comprises, in a first stage (i), reacting methane to form ethylene and, in a later stage (ii), reacting the product mixture obtained in stage (i) which comprises ethylene and methane to give higher-value hydrocarbons.

In addition, the present invention relates to a plant for producing hydrocarbons from methane in which, in a single plant strand, a plurality of plant units are arranged successively in series comprising:
a first reactor A for carrying out a conversion from methane to ethylene
a second reactor B for carrying out a conversion from ethylene to higher-value hydrocarbons.

The present invention relates to a process for producing hydrocarbons from methane, which comprises, in a first stage (i), reacting methane to form ethylene and, in a later stage (ii), reacting the product mixture obtained in stage (i) which comprises ethylene and methane to give higher-value hydrocarbons.

In addition, the present invention relates to a plant for producing hydrocarbons from methane in which, in a single plant strand, a plurality of plant units are arranged successively in series comprising:
a first reactor A for carrying out a conversion from methane to ethylene
a second reactor B for carrying out a conversion from ethylene to higher-value hydrocarbons.

One of the great challenges in the field of heterogeneous catalysis is the conversion of methane into more valuable chemicals and fuels. Particular importance is attached to the reaction to form ethylene which is obtainable by coupling methane. Methane coupling to form ethylene can be performed, for example, by oxidation or by oligomerization.

The expression coupling is generally taken to mean those reactions in which two organic molecules are connected to one another, forming a bond between carbon atoms or a carbon atom and a heteroatom.

In the case of methane coupling, methane is first converted to ethane and thereafter to ethylene. The reaction system is complex since it comprises not only a heterogeneous part, the activation of CH₄ at a metal oxide surface, but also a homogeneous free radical reaction in the gas phase. Ethane is formed principally from CH₃ radicals which are formed on the catalyst surface and dimerize in the gas phase. The yield of C₂H₄ and C₂H₆ is limited by side reactions of the CHs-free radicals with the surface and by further reaction of C₂H₄ both at the catalyst surface and also in the gas phase.

Catalysts which are disclosed for coupling methane are, for example, oxides such as magnesium oxide, calcium oxide, strontium oxide, silica oxide, manganese oxide, aluminum oxide, tungstate manganese oxide on silica oxide, lanthanum oxide, cerium oxide, sammarium oxide, europium oxide, lithium on magnesium oxide and yttrium oxide, furthermore iron-oxide-doped zeolites or reducible oxides such as, for example, doped or undoped manganese oxide. The catalytic reaction is typically carried out at a temperature of 400 to 1000° C. and at atmospheric pressure up to pressures of 100 bar. The yield of ethylene can reach up to 50%, wherein the ethylene selectivity is up to 100%.

U.S. Pat. No. 6,037,514 describes coupling of methane in a membrane reactor, wherein the oxidizing agent oxygen is separated from methane by the membrane. The catalyst used was BaCe_{0.9}Mn_{0.1}O_{3-y}. The reaction was carried out at 950° C. An ethylene production rate of 0.2 ml (STP) min⁻¹ cm⁻² is described, wherein the selectivity was approximately 100%.

GB 2 252 104 describes oxidative coupling of methane using MnO₂/25% KCl as catalyst. The oxidizing agent used was oxygen. The reaction was carried out at 750° C. and a C2 selectivity greater than 90% was obtained. The ethylene selectivity was greater than 80%. The conversion rate was approximately 20%.

In J. Catal. 117 (1989) 362-370, oxidative coupling of methane in a quartz reactor is described, wherein an iron-oxide-doped zeolite was used as catalyst. The reaction was carried out at 400° C. and atmospheric pressure. The oxidizing agent used was N₂O. The ethylene selectivity was greater than 90%, and the methane conversion rate was 5 to 10%.

In Theoretical and Experimental Chemistry 41 (2005) 117-121, again, oxidative coupling of methane in a quartz reactor is described, wherein the catalyst used was SrNi_{0.75}Li_{0.25}O₃₋ₓ. The reaction was carried out at 700° C. and atmospheric pressure. The oxidizing agent used was oxygen. The C2-C4 selectivity achieved was greater than 99.5%, wherein the ethylene selectivity was 65%. The methane conversion rate was greater than 22%.

Despite extensive research work, there is currently still no industrial process for methane coupling. Presumably, the low methane conversion rate and the associated high costs of separating off the small amount of ethylene in the product stream prevent commercialization.

However, there is great economic interest in commercializing methane coupling. As a result of a commercially feasible methane coupling, natural gas could be used not only as an energy source, but also increasingly as a key building block for producing many valuable key chemicals.

Natural gas is currently principally used as a primary energy source; as a chemical feedstock natural gas still plays a small role. The reliably obtainable reserves worldwide of natural gas are in the order of magnitude of around 5000 EJ. Assuming that production remains the same, the reserves would last for at least a further 50 years.

Currently, petroleum (naphtha) is the most important key building block for producing a large number of chemical products. The petrochemicals ethylene, propylene, butenes and the aromatics benzene, toluene and xylene which occur on cracking and reforming petroleum serve as feedstocks for a multiplicity of products.

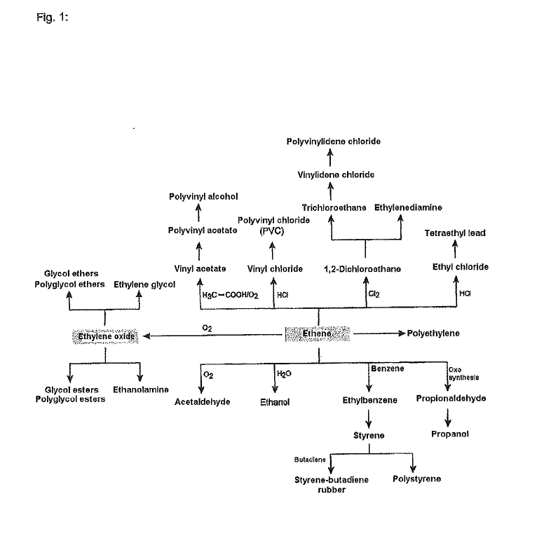

Ethylene is one of the most important products of petrochemistry and feedstock for a multiplicity of products. FIG. 1 gives an overview of the importance of petroleum processing products from ethylene.

Ethylene was originally obtained by partial hydrogenation of carbide acetylene, by dehydration of ethanol or by isolation from coke oven gas. These processes are now of no importance in countries having a developed petrochemical industry. Ethylene is now solely obtained from petroleum and natural gas by thermal cracking. In Western Europe, for a long time there has been insufficient ethane-rich natural gas, so that here as also in some other countries, e.g. in Japan, naphtha was used as the chief olefin raw material. Increasing use of natural gas from domestic gas sources and also from imports have allowed the naphtha fraction to decrease in Western Europe to currently about 76%. Disadvantages of the cracking process, however, are the downstream extensive purification and gas separation processes which are necessary until the ethylene has the purity necessary for further processing; the removal of acetylene requires particular effort.

Shortages in petroleum production as a result of natural exhaustion or political manipulations can have very far reaching consequences for supplying the producing chemical industry with petrochemicals. In view of the constantly increasing requirement for petrochemicals, worldwide not only is research for novel production methods being intensified, but there are also efforts to recover free petroleum capacities for purposes of petrochemistry by exploiting other energy sources and raw materials (nuclear energy, solar energy and hydroenergy, recycling of waste, reprocessing of used oil).

There was consequently a need for an alternative raw material to petroleum for producing important base chemicals. At best this raw material will be more expedient and have a higher availability than petroleum. In addition there is interest in finding an alternative process to the familiar cracking processes which at best will deliver direct access to the petrochemicals and/or other base chemicals from natural resources.

In addition, there is economic interest in commercializing methane coupling, since by means of possible conversion of methane, the currently high transport costs of natural gas could be decreased.

Methane occurs in large amounts as natural gas, the reserves of which are distributed around the world. These natural gas deposits are frequently very far away from industrial areas and/or the end users, for example the large natural gas fields in Alaska or Siberia. It follows therefrom that natural gas must be transported over long distances. Owing to the gaseous nature of natural gas, transport is difficult. Although natural gas can be liquefied at very low temperatures and high pressures, this means a significant cost factor which can possibly be decisive in the decision about exploitation of a new natural gas field.

Consequently, the transport costs could be reduced if natural gas/methane is converted directly at the respective deposit into a higher hydrocarbon which is already liquid or can be liquefied more readily than methane.

A known process is converting methane into a mixture of carbon monoxide and hydrogen, called synthesis gas, and its further reaction into diesel or other transportable fuels. A disadvantage, however, is that this conversion involves not only high capital costs but also high running reaction costs. In addition, the greenhouse gas carbon dioxide is produced and consequently the carbon selectivity is low.

The literature concerned with reducing the high transport costs of methane via conversion to higher hydrocarbons, unfortunately, contains no indication of the use of methane coupling.

Because of the high requirement of ethylene for producing important base chemicals such as, for example, ethylene oxide, polyethylene, ethylbenzene, ethanol, acetaldehyde, propionaldehyde, vinyl acetate, vinyl chloride, 1,2-dichloroethane and/or ethyl chloride, there is, in addition, great interest in being able to use commercially methane coupling in the production of these base chemicals.

It would be of very great economic interest if it were possible to use the product mixture which is obtained from methane coupling and comprises methane, ethane, ethylene and small amounts of COₓ as a starting product mixture without costly separation steps in a process for producing higher-value hydrocarbons. Consequently, despite the low conversion rate of methane in the methane coupling, an economic process could be developed.

In order to find an economic linkage, methane and possibly ethane as well should not significantly influence the subsequent reaction to give higher-value hydrocarbons.

Currently, however, methane is rarely used as an accompanying gas or inert gas. Noble gases and, in particular, inexpensive nitrogen, are principally used as inert gases, since nitrogen can neither be decomposed nor reacted.

In the literature on production of ethylene oxide, however, there is the indication that methane can advantageously be used as an inert gas.

The outstanding importance of ethylene oxide which has an annual production of 15 million tons is in the reactivity of the oxirane ring which makes it a key substance for a multiplicity of further intermediates and end products. Ethylene oxide is principally used for producing ethylene glycol and its derivatives, ethanolamines, and also polyols, polyethylene glycols and ethoxylates, which are used in emulsifiers, laundry detergents and wetting agents.

Ethylene oxide is frequently produced industrially by direct oxidation of ethylene with oxygen in the presence of silver-containing catalysts at 200 to 300° C. and 1.5 to 2.0 MPa. The starting material mixture of ethylene and oxygen in addition comprises methane and possibly steam, carbon dioxide, halogen compound and/or nitrogen compounds.

Typically, the starting material ethylene originates from steam-cracking processes, for example steam cracking of oil or naphtha or steam cracking of ethane which occurs as an accompanying gas in petroleum or natural gas extraction. Likewise, the ethylene can also originate from a catalytic, oxidative or autothermal dehydrogenation of ethane. In addition, the European patent application having the application number 08166057.3 describes that use can be made of ethylene which is obtained by dehydration of ethanol.

No literature references describe making use of the product mixture comprising ethylene and methane from methane coupling as a starting material/inert gas mixture in the production of ethylene oxide.

The object of the present invention is therefore to demonstrate a commercialization of methane coupling.

A further object of the present invention is to provide a process in which methane or natural gas is the starting point for producing higher-value hydrocarbons and natural gas is thereby used via the methane coupling to form ethylene as a building block for valuable chemicals.

In particular, the object of the present invention was to demonstrate an economical process for producing ethylene oxide from methane or natural gas.

Surprisingly, it has been found that a combination of hitherto uneconomical methane coupling with a process for producing higher-value hydrocarbons is a much promising process for producing ethylene oxide, for example, from natural gas and thereby a starting product for numerous chemical reactions.

The present invention therefore relates to a process for producing higher-value hydrocarbons from methane, which comprises, in a first stage (i), reacting methane to form ethylene and, in a later stage (ii), reacting the product mixture obtained in stage (i) which comprises ethylene and methane to give higher-value hydrocarbons.

The expression “higher-value hydrocarbons” is taken to mean, in the present invention, the hydrocarbons which have a higher market value than ethylene. The market value may be derived, for example, on the basis of the respective prices for the hydrocarbons. The higher-value hydrocarbons can have, in addition to the atoms C and H, further heteroatoms such as, for example, O, S, P and N. In addition, the higher-value hydrocarbons have a higher molecular weight than ethylene. By way of example, ethylene oxide, polyethylene, ethylbenzene, ethanol, acetaldehyde, propionaldehyde, vinyl acetate, vinyl chloride, 1,2-dichloroethane and/or ethyl chloride are higher-value hydrocarbons.

In general, in stage (i), the reaction of methane to form ethylene, of the process according to the invention, use can be made of all methane coupling processes known to those skilled in the art. Advantageously, methane coupling is carried out as oxidative methane coupling or as methane oligomerization. By way of example, such processes are described in U.S. Pat. No. 6,037,514 (methane oligomerization), GB 2 252 104, J. Catal. 117 (1989) 362-370, and in Theoretical and Experimental Chemistry 41 (2005) 117-121 (oxidative methane coupling).

For example, the reaction of methane to form ethylene in stage (i) can be carried out catalytically. Advantageously, this catalyst comprises one or more of the following materials: Al, Ba, Ca, Ce, Eu, Fe, Ga, K, La, Li, Mg, Mn, Na, Si, Sm, Sr, W and/or Y.

As methane source of stage (i), use can be made of all methane sources known to those skilled in the art. Advantageously, natural gas is used as methane source, for example the methane required originates directly from a natural gas pipeline or from a natural gas source. Preferably, the natural gas is used directly for stage (i), i.e. without any separation or purification stages. Additives known to those skilled in the art can optionally be admixed with the methane or the methane-comprising natural gas; these additives can optionally also be added separately to the reactor of stage (i).

As natural gas, all natural gas fields come into consideration. Natural gas usually comprises an alkane mixture. The alkane mixture comprises as main component methane and in smaller amounts a mixture of longer-chain alkanes, in particular C₂-C₆ alkanes. For example, the natural gas comprises the following mixture:
75 to 99%, preferably 80 to 98%, in particular 85 to 97%, methane, based on the natural gas;
1 to 15%, preferably 1 to 10%, in particular 1 to 7%, ethane, based on the natural gas;
0.1 to 10%, preferably 0.1 to 5%, in particular 0.1 to 3%, propane, based on the natural gas;
0 to 3%, in particular 0.01 to 2%, butane (n-butane and/or isobutane), based on the natural gas;
0 to 2%, preferably 0.01 to 1%, pentane (n-pentane and/or isopentane), based on the natural gas;
0 to 1%, preferably 0.01 to 0.1%, hexane, based on the natural gas.

Stage (i) can be carried out in all reactors known therefor to those skilled in the art. For example, stage (i) can be carried out in one or more fixed bed reactors, fluidized bed reactors, membrane reactors, microchannel reactors and/or combinations thereof. Advantageously, stage (i) is carried out using the oxidative methane coupling in one or more fixed bed(s) and/or in one or more membrane reactor(s) and using methane oligomerization in one or more membrane reactor(s).

Advantageously, stage (i) is carried out at a temperature from 100 to 1100° C., preferably from 200 to 1000° C., particularly preferably from 350 to 950° C.

Advantageously, stage (i) is carried out at a pressure from 0 to 100 bar, preferably from 0 to 60 bar, particularly preferably from 0 to 50 bar in particular from 5 to 50 bar, most particularly preferably from 5 to 20 bar.

The conversion rate of the reaction in stage (i) is typically 5 to 80%, preferably 10 to 60%, in particular 15 to 50%.

The ethylene selectivity is advantageously 10 to 100%, preferably 50 to 100%, in particular 60 to 100%.

The C₂ selectivity is advantageously 30 to 100%, preferably 50 to 100%, in particular 60 to 100%.

The C₂₊ selectivity is advantageously 40 to 100%, preferably 50 to 100%, in particular 60 to 100%, wherein the expression C₂₊ comprises not only C₂ but also longer-chain hydrocarbons.

Advantageously, the conversion of stage (i) is carried out catalytically. As catalyst of stage (i), use can be made of all catalysts known to those skilled in the art,

The product mixture (a) obtained from stage (i), the methane coupling, advantageously comprises at least 8 vol.-% of ethylene, less than 80% of methane and less than 20 vol.-% of COₓ, based on the carbonaceous components of the product mixture (a). Preferably, the product mixture (a) comprises 5 to 30 vol.-% of ethylene, 0 to 30 vol.-% of ethane, 40 to 80% of methane and 0 to 10% of COₓ, based on the carbonaceous components of the product mixture (a).

When oxidative methane coupling is used, as oxidizing agents of stage (i), use can be made of all oxidizing agents known to those skilled in the art. When a fixed bed reactor is used, advantageously oxygen is used as oxidation source. The required amount of oxygen can be fed to the reactor A by all means known to those skilled in the art, for example the entire amount of oxygen required is fed at the reactor entry or fed at a plurality of reactor positions distributed over the reactor. When a membrane reactor is used, advantageously air is used as oxidation source.

A separation unit T1.1 can optionally be integrated between stage (i) and stage (ii). This separation unit advantageously separates off ethane from the product mixture (a). Alternatively, only a substream of the product mixture (a) can pass through the separation unit T1.1. Separating off ethane can proceed using all separation techniques known to those skilled in the art, for example by pressure-swing adsorption, by membranes or by distillation. The ethane which is separated off is advantageously fed to the methane and/or methane-comprising natural gas.

Instead of the separation unit T1.1 or additionally to this, a reactor (DH) for the (oxidative) dehydrogenation of ethane to ethylene can optionally be integrated between stage (i) and stage (ii). Alternatively, only a substream of the product mixture (a) can pass through the dehydrogenation reactor. The dehydrogenation of ethane can proceed using all techniques known to those skilled in the art.

A further or alternative separation unit T1.2 can optionally be integrated between stage (i) and stage (ii). This separation unit advantageously separates off C₃₊ hydrocarbons from the product mixture (a). Alternatively, only a substream of the product mixture (a) can pass through the separation unit T1.2. Separating off C₃₊ hydrocarbons can proceed using all separation techniques known to those skilled in the art, for example by pressure-swing adsorption, by membranes or by distillation.

A separation unit T1.3 for separating off CO₂ can optionally be integrated between stage (i) and stage (ii). Alternatively only a substream of the product mixture (a) can pass through the separation unit T1.3. Separating off CO₂ can proceed using all techniques known to those skilled in the art, preferably a CO₂ scrubber is used.

The product mixture (a) obtained from stage (i) can be passed into the following reactor B for carrying out the conversion of the ethylene to higher-value hydrocarbons by all techniques known to those skilled in the art.

Advantageously, the product mixture (a) obtained in stage (i) is used as starting material for stage (ii) without separating off or reducing the methane content of the product stream.

Additives known to those skilled in the art can optionally be admixed with the product mixture (a) from stage (i) or can be fed to the reactor B separately from this product mixture.

For example, in the production of ethylene oxide, steam, carbon dioxide, halogen compound and/or nitrogen compounds as described, for example, in European patent application having the application number 08166057.3 can be admixed to the product mixture (a).

The production of higher-value hydrocarbons can be carried out by all processes known to those skilled in the art. Advantageously, in stage (ii), an oxidation, an epoxidation, a hydration, an oxo synthesis, a halogenation and/or a polymerization is carried out. Advantageously, the process of stage (ii) is carried out by catalysis. For example, the higher-value hydrocarbons produced are ethylene oxide, polyethylene, ethylbenzene, ethanol, acetaldehyde, propionaldehyde, vinyl acetate, vinyl chloride, 1,2-dichloroethane and/or ethyl chloride. Preferably, the higher-value hydrocarbon produced is ethylene oxide.

For example, the reaction of stage (ii) in the production of ethylene oxide can be carried out in one or more fixed bed reactors, fluidized-bed reactors, membrane reactors, microchannel reactors and/or combinations thereof.

Advantageously, in the exemplary case of producing ethylene oxide, the oxidation of stage (ii) is carried out at a temperature of 50 to 400° C., preferably from 100 to 350° C., particularly preferably from 150° C. to 300° C.

Advantageously, the ratio (Tᵢ/Tᵢᵢ) of the temperature (Tᵢ) of the reaction of stage (i) to the temperature (Tᵢᵢ) of the reaction of stage (ii) is between 1 and 5, preferably between 1 and 4, in particular between 1 and 3.

Advantageously, in the exemplary case of producing ethylene oxide, the oxidation of stage (ii) is carried out at a pressure of 0 to 100 bar, preferably from 0 to 80 bar, particularly preferably from 0 to 60 bar in particular from 5 to 50 bar, most particularly preferably from 5 to 20 bar.

The pressure difference between the reactions of stages (i) and (ii) is advantageously less than 10 bar, preferably less than 5 bar, in particular less than 3 bar.

Advantageously, in the exemplary case of producing ethylene oxide, the GHSV (gas hourly space velocity) is 1000 to 20 000 h⁻¹, preferably 2000 to 10 000 h⁻¹.

Advantageously, in the exemplary case of producing ethylene oxide, use is made of a catalyst, the active catalyst mass of which comprises silver, one or more alkali metal compounds and possibly one or more co-promoters selected from the group consisting of sulfur, molybdenum, tungsten, chromium and rhenium.

Advantageously, a supported catalyst is used. The support used is advantageously α-aluminum oxide.

Particularly preferably, in the stage (i), methane is catalytically converted to ethylene, wherein the catalyst of the stage (i) is one or more of the following materials: Al, Ba, Ca, Ce, Eu, Fe, Ga, K, La, Li, Mg, Mn, Na, Si, Sm, Sr, W and/or Y, and, in the stage (ii), ethylene is catalytically converted to ethylene oxide, wherein the catalyst used is a catalyst supported on a-aluminum oxide, the active mass of which comprises silver, one or more alkali metal compounds and optionally one or more co-promoters selected from the group consisting of sulfur, molybdenum, tungsten, chromium and rhenium.

Advantageously, the product mixture (b) from stage (ii) comprising higher-value hydrocarbon, methane, carbon dioxide, possibly ethane and small amounts of ethylene is separated into a product stream (b1) comprising higher-value hydrocarbon and a product stream (b2) comprising methane, carbon dioxide, possibly ethane and possibly small amounts of ethylene. This separation (T2) can proceed by all techniques known to those skilled in the art, for example by pressure-swing adsorption, by membranes or by distillation.

Advantageously, the product stream (b2) thus obtained is passed to a power station or back into the natural gas pipeline without further separation or purification of the product stream. Recycling to the natural gas pipeline is particularly advantageous when the process according to the invention is carried out throughout at elevated pressure of approximately 40 to 70 bar. The power station can utilize this product stream (b2), for example for energy production.

In addition, advantageously, the product stream (b2) can be further separated. Advantageously, the product stream (b2) can be separated into a product stream (c1) comprising ethylene and possibly small amounts of carbon dioxide, and a product stream (c2) comprising methane, carbon dioxide, possibly ethane and possibly small amounts of ethylene. This separation (T3) can be performed by all techniques known to those skilled in the art, for example by pressure-swing adsorption, by membranes or by distillation.

The product stream (c2) can then advantageously, as described above, be passed into a power station or back into the natural gas pipeline.

The product stream (c1) advantageously passes through a further separation stage (T4) in which the possibly small amounts of carbon dioxide are separated off. This separation can again proceed by all techniques known to those skilled in the art, for example by pressure-swing adsorption, by membranes, by distillation or by CO₂ gas scrubbing. The product stream (c3) obtained after the separation which comprises ethylene is advantageously admixed to the product mixture (a) from stage (i) upstream of entry into the reactor B of stage (ii) or fed to the reactor B separately from the product mixture (a).

Advantageously, consequently, the ethylene obtained in stage (i) which is not reacted in the reactor B is recirculated.

Advantageously, the process according to the invention is carried out continuously.

In addition, the present invention relates to a plant for producing higher-value hydrocarbons from methane in which, in a single plant strand, a plurality of plant units are arranged successively in series comprising:
a first reactor A for carrying out a conversion from methane to ethylene
a second reactor B for carrying out a conversion from ethylene to higher-value hydrocarbons.

Advantageously, the reactor A serves for carrying out a methane coupling, in particular a catalytic oxidative methane coupling or a catalytic methane oligomerization. The reactor A advantageously has feed lines for the methane source, in particular for natural gas, and optionally for the oxygen source or further additives.

Advantageously, the reactor B serves for carrying out a catalytic conversion of ethylene to higher-value hydrocarbons, for example ethylene oxide, polyethylene, ethylbenzene, ethanol, acetaldehyde, propionaldehyde, vinyl acetate, vinyl chloride, 1,2-dichloroethane and/or ethyl chloride, preferably ethylene oxide. The reactor B is advantageously connected to the reactor A via feed lines. In addition, the reactor B has feed lines for an oxygen source and optionally further additives.

Between the reactor A and B there are optionally a separation unit T1.1 for separating off ethane, and/or a reactor DH for dehydrogenating ethane to ethylene, a separation unit T1.2 for separating off C₃₊ hydrocarbons and/or a separation unit T1.3 for separating off CO₂.

Downstream of the reactor B, a separation unit T2 for separating off the higher-value hydrocarbons can advantageously be situated.

In addition, the plant according to the invention can have a third reactor C which advantageously serves for energy production.

Downstream of the separation unit T2 there can optionally be situated a further separation unit T3 for separating off the unreacted ethylene.

Advantageously, the unreacted ethylene is separated off in a further separation unit T4 from the carbon dioxide remaining in the product stream (c1).

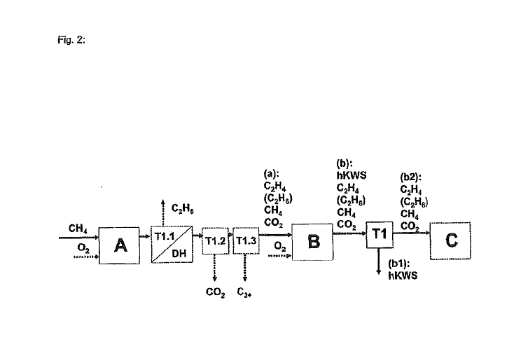

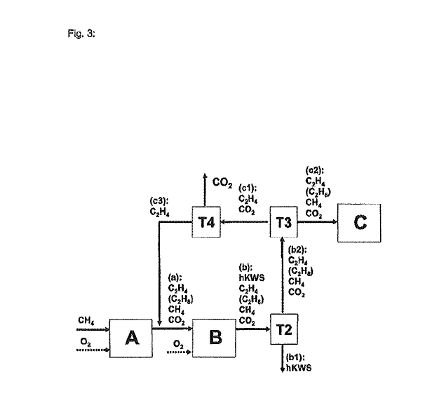

The reaction sequence is shown schematically in accompanying FIGS. 2 and 3, wherein hKWS is higher-value hydrocarbons. The optionality of some separation units/reactors is indicated by dashed lines.

Ethylene oxide is a key substance for a multiplicity of further intermediates and end products.

Ethylene oxide serves, for example, as starting material for producing ethylene glycol by reaction with water, for producing ethanolamines by reaction with ammonia, for producing alkyl- and/or arylalkanolamines by reaction with alkyl- or arylamines, for producing (alkyl)phenol oxethylates with (alkyl)phenol, for producing glycol ethers with alcohol, for producing alcohol oxethylates with alcohol, for producing polyethylene glycol with ethylene oxide and for producing polyalkylene glycol with alkylene oxides.

The process according to the invention for producing higher-value hydrocarbons from methane is the first economic use of methane coupling. By means of the combination according to the invention of the conversion of methane to ethylene and of ethylene to higher-value hydrocarbons, in particular ethylene oxide, a reasonable economic efficiency can be achieved, since in the direct process according to the invention, any separation of methane can be omitted.

In addition, it is advantageous that the unreacted methane can be used directly for energy production, similar to direct energy production from natural gas, or can be conducted back into the natural gas pipeline.

The coupling of two processes having high selectivity enables optimal utilization of the ethylene formed in step (i).

It is also advantageous that the process according to the invention can also be integrated into existing ethylene oxide, polyethylene, ethylbenzene, ethanol, acetaldehyde, propionaldehyde, vinyl acetate, vinyl chloride, 1,2-dichloroethane and/or ethyl chloride plants.

### EXAMPLE

### Process for Producing Ethylene Oxide:

From an ethylene selectivity of greater than 50% in stage (i), on account of the high price difference between methane and ethylene oxide having a factor 1:5, the process according to the invention is economic. The capital costs are taken into account.

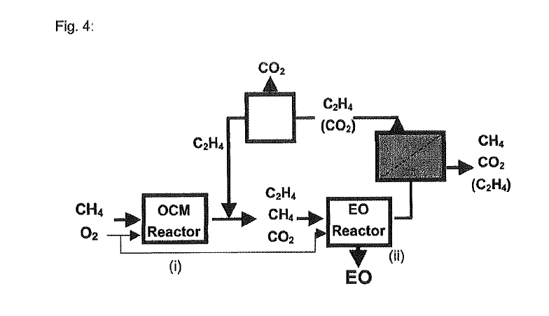

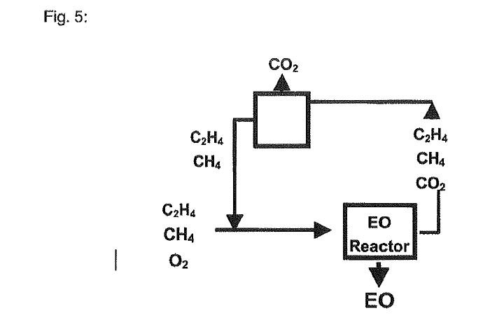

The economic efficiency of the process according to the invention is illustrated in the following two examples. An ethylene oxide capacity of 350 kt ethylene oxide per year is assumed. The selectivities described in the prior art of the stage (i) (methane to ethylene) are between 0.5 and 0.99, in addition the described selectivities of the stage (ii) (ethylene to ethylene oxide) are between 0.7 and 0.9. The process according to the invention is shown schematically in FIG. 4. The process according to the invention is compared with the conventional process (as bench mark). The conventional process is shown schematically in FIG. 5.

### Example 1

For a selectivity of ethylene of 0.8 in the stage (i) and a selectivity of ethylene oxide of 0.85 in the stage (ii), this gives a savings of more than [symbol] 100 per tonne of ethylene oxide. This corresponds to a savings of more than [symbol] 35 million per year (at an assumed capacity of 350 kta of ethylene oxide).

### Example 2

At a selectivity of ethylene of 0.9 in the stage (i) and a selectivity of ethylene oxide of 0.87 in the stage (ii), this gives a savings of more than [symbol] 220 per tonne of ethylene oxide. This corresponds to a savings of more than [symbol] 75 million per year (at an assumed capacity of 350 kta of ethylene oxide).

1.-15. (canceled)

16. A process for producing ethylene oxide from methane, which comprises, in a first stage (i), reacting methane to form ethylene and, in a later stage (ii), using the product mixture (a) obtained in stage (i) which comprises ethylene and methane and reacting ethylene to give ethylene oxide and carrying out the reactions of the stages (i) and (ii) at a pressure range from 5 bar to 50 bar.

17. The process according to claim 16, wherein the product mixture obtained in stage (i) is used without separating off methane or any other reduction of the methane content as starting material mixture for stage (ii).

18. The process according to claim 16, wherein the pressure difference between the reactions of the stages (i) and (ii) is less than 10 bar.

19. The process according to claim 16, wherein the ratio (Tᵢ/Tᵢᵢ) of the temperature (Tᵢ) of the reaction of the stage (i) to the temperature (Tᵢᵢ) of the reaction of the stage (ii) is between 1 and 4.

20. The process according to claim 16, wherein the methane originates from a natural gas pipeline or a natural gas source.

21. The process according to claim 16, wherein the higher-value hydrocarbons are separated off after stage (ii) and the obtaining residual gas stream is passed into a power plant or back into the natural gas pipeline.

22. The process according to claim 16, wherein the process is carried out continuously.

23. The process according to claim 16, wherein the reaction in stage (i) is carried out as catalytic oxidative methane coupling or as catalytic methane oligomerization.

24. The process according to claim 16, wherein, in the stage (i), methane is catalytically reacted to form ethylene and the catalyst of the stage (i) comprises one or more of the following materials: Al, Ba, Ca, Ce, Eu, Fe, Ga, K, La, Li, Mg, Mn, Na, Si, Sm, Sr, W and/or Y and in stage (ii) ethylene is catalytically reacted to form ethylene oxide and the catalyst used is a catalyst supported on a-aluminum oxide, the active mass of which comprises silver, one or more alkali metal compounds and optionally one or more co-promoters selected from the group consisting of sulfur, molybdenum, tungsten, chromium and rhenium.

25. A system for producing ethylene oxide from methane in which, in a single plant strand, a plurality of plant units are arranged successively in series comprising:
a first reactor A for carrying out a conversion from methane to ethylene
a second reactor B for carrying out a conversion from ethylene to ethylene oxide,
the plant strand comprising the following further separation units or reactors:
a separation unit T1.1 for separating off ethane or a reactor DH for dehydrogenation of ethane to ethylene, arranged between the reactors A and B of stages (i) and (ii)
a separation unit T1.2 for separating off CO₂, arranged between the reactors A and B of stages (i) and (ii)
a separation unit T2 for separating off ethylene oxide, arranged downstream of the reactor B of stage (ii)
a separation unit T3 for separating off ethylene, arranged downstream of the separation unit T2.
